(19) United States
(12) Patent Application Publication
(10) Pub. No.: US 2023/0097323 A1
(43) Pub. Date: Mar. 30, 2023

(54) Method and Devices for Transdermal Delivery of Hydrogen Gas for Therapeutic Purposes

(71) Applicant: Hyderm Innovations LLC, Dallas, TX (US)

(72) Inventors: Vladimir KLICHKO, Dallas, TX (US); Svetlana RADYUK, Dallas, TX (US); Yaroslav KLICHKO, San Jose, CA (US); Daniel CROW, Dallas, TX (US)

(21) Appl. No.: 17/863,234

(22) Filed: Jul. 12, 2022

Related U.S. Application Data
(60) Provisional application No. 63/245,138, filed on Sep. 16, 2021.

(51) Int. Cl. A61K 9/70; A61K 33/00

(52) U.S. Cl. CPC A61K 9/7084; A61K 33/00

(57) ABSTRACT

Molecular hydrogen has been shown to have cosmetic and medicinal benefits. However, delivery methods have been ineffective in aiding the body in receiving and absorbing the hydrogen. This device has a dry chemical separated from a solution. Both the dry chemical and the solution are housed in a container. The container has a hydrogen impermeable top outer layer and a hydrogen permeable bottom layer. Once initiated, a determined and controlled chemical reaction generates molecular hydrogen within the container. The hydrogen then exits the device through the hydrogen permeable side of the device that is facing the skin. The device carries a low risk of toxicity while providing cosmetic and medicinal advantages.

### PRIORITY

The present application claims priority to U.S. Provisional Application No. 63/245,138, filed Sep. 16, 2021, the entirety of which is hereby incorporated by reference.

### BACKGROUND OF THE INVENTION

### Technical Field

The present invention relates to a system and method for transdermal delivery of hydrogen gas for therapeutic purposes. A device in the form of a patch, bag, bandage, wrap or other container for delivering hydrogen gas to a target area of the body for therapeutic treatment. The device remains inert until activated by breaking the internal inserts that separate the chemicals, resulting in the formation of molecular hydrogen, which is released from the device and diffused into the target area of the body.

### Description of Related Art

Molecular hydrogen (H₂) possesses strong antioxidant, anti-inflammatory and other protective effects on organs and tissues. Additionally, hydrogen has no known adverse or toxic effects on cell function. Hydrogen molecules easily penetrate into cells and can be delivered to a wide range of body organs and tissues. Although a significant amount of this gas is produced by the bacteria of the host colon under physiological conditions, exogenously applied H₂ can have a significant effect on physiology and functions of the body and is used to treat various pathological conditions and diseases and restore normal physiology and functions (Reviewed in Shen M, Zhang H, Yu C, Wang F, Sun X. A review of experimental studies of hydrogen as a new therapeutic agent in emergency and critical care medicine. Med Gas Res. 2014; 4:17; Ohno K, Ito M, Ichihara M, Ito M. Molecular hydrogen as an emerging therapeutic medical gas for neurodegenerative and other diseases. Oxid Med Cell Longev. 2012; 2012:353152; Huang C S, Kawamura T, Toyoda Y, Nakao A. Recent advances in hydrogen research as a therapeutic medical gas. Free Radic Res. 2010; 44(9):971-82; Ge L, Yang M, Yang N N, Yin X X, Song W G. Molecular hydrogen: a preventive and therapeutic medical gas for various diseases. Oncotarget. 2017; 8(60):102653-73; Ohta S. Molecular hydrogen as a novel antioxidant: overview of the advantages of hydrogen for medical applications. Methods Enzymol. 2015; 555:289-317). As the benefits of hydrogen are numerous, many methods have been developed to deliver exogenous hydrogen into the body.

Externally produced hydrogen can be administered by inhalation in the form of gas, and by injections or oral intake of hydrogen-saturated solutions. The former method suffers from safety issues and is only feasible in the laboratory environment or at special facilities, but not in routine medical practice or at home. Oral intake also requires large volumes of H₂-rich liquids to reach the desired H₂ concentrations in the body organs and tissues. Besides, hydrogen-rich water and solutions cannot be stored for a prolonged period of time. Administration of hydrogen by injection is invasive and is applicable at a specific setting and by medical personnel. Disadvantages of all of these methods makes the therapy cost prohibitive.

Alternatively, hydrogen can be produced in vivo by oral uptake of H₂-generating chemicals such as metallic Mg that reacts with acidic gastric environment and produces H₂. This approach suffers from potential toxicity of products (e.a.magnesium hydroxide) formed in the reaction.

Hydrogen gas can also be delivered transdermally by soaking the body in a bath with H₂-saturated water or by applying hydrogen-producing chemical compositions to the skin. Another form in which hydrogen was introduced is atomic hydrogen surrounded by water molecules, H(H₂O)m, generated by the device where a pulse-shaped voltage is applied between the inner and outer electrodes. The device is applied to the skin for the topical treatment of various skin damages. However, these methods suffer from many drawbacks. Thus, soaking the body requires large volumes of H₂-rich water and suffers from a short duration of treatment due to rapid evaporation of H₂; the H(H₂O)m-based method requires complex equipment, which then makes the therapy cost prohibitive; the use of hydrogen gas-producing chemical compositions that are applied directly to the skin can cause many undesirable effects such as irritation, redness, burning and rashes, as well as suffer from the potential toxicity of the products formed in the process of H₂-producing reaction.

Although the administration of H₂ by inhalation or ingestion is the predominant method and is capable of providing systemic effects, delivery of molecular hydrogen through the skin can provide targeted delivery to sites of interest for a more efficient local effect due to the H₂ concentration gradient. For example, inhaled H₂ mainly affects the respiratory system, while hydrogen introduced orally acts primarily through receptors in the gastro-intestinal system. Thus, transdermal delivery differs from other methods of hydrogen administration in its effect on a specific area of the body. Consequently, the widespread use of hydrogen-based approaches has been limited due to the obstacles associated with its availability and application. Therefore, there is a need in the art for a new device/method for delivering molecular hydrogen to a specific area of the body.

### BRIEF DESCRIPTION OF THE DRAWINGS

The novel features believed characteristic of the invention are set forth in the appended claims. The invention itself, however, as well as a preferred mode of use, further objectives and advantages thereof, will be best understood by reference to the following detailed description of illustrative embodiments when read in conjunction with the accompanying drawings, wherein:

FIG. 1 shows a top view of a hydrogen-producing system/device according to various embodiment described herein.

FIG. 2 depicts an exploded perspective view of one example of a hydrogen producing system/device according to various embodiments described herein.

FIG. 3 shows a bottom perspective view of the system/device according to various embodiments described herein.

FIG. 4 depicts a cross-sectional perspective view of the system/device according to various embodiments described herein.

FIG. 5A shows a top view, and FIGS. 5B and 5C depict side views of the system/device according to various embodiments described herein.

FIG. 6 depicts a top view of an alternative activation system for the device according to various embodiments described herein.

FIG. 7 depicts a cross-sectional perspective view of the system/device according to various embodiments described herein.

FIG. 8 depicts an exploded perspective view of one example of a hydrogen producing system/device according to various embodiments described herein.

FIG. 9A-9D illustrates a perspective views of examples of a patch on other parts of the body according to various embodiments described herein;

FIG. 10 depicts an embodiment of system/device as a wrap that is placed around an entire portion of a body according to various embodiments described herein;

FIG. 11 illustrates an embodiment wrapped around a leg of the system/device according to various embodiments described herein.

### DETAILED DESCRIPTION

Several embodiments of Applicant's invention will now be described with reference to the drawings. Unless otherwise noted, like elements will be identified by identical numbers throughout all figures. The invention illustratively disclosed herein suitably may be practiced in the absence of any element which is not specifically disclosed herein.

To overcome the shortcomings of the prior art, we have developed a method/device that supplies molecular hydrogen gas generated inside a sealed bag upon activation. In one embodiment the bag is plastic. Delivery is controlled, while limiting direct contact of the hydrogen-generating reagents with the skin. It is believed that H₂ enters the cells and subcellular compartments of various tissues by gas diffusion. Thus, hydrogen is supplied to the target tissue/organ by diffusion from inside the device containing a hydrogen-generating mixture to the body. The disclosed approach is free of all mentioned disadvantages. In one embodiment, the device contains all chemicals and solutions separated within the container and therefore cannot cause any side effects in their inert state. Instead, in such embodiments, only molecular hydrogen, formed during a chemical reaction inside the package or released from H₂-accumulating components, then diffuses through the hydrogen-permeable surface and penetrates into and through the skin. In addition, since a long supply of hydrogen is required for effective treatment, this device enables the production and stable delivery of hydrogen for a sufficient duration.

The device and method discussed herein can be used to treat both accurate and chronic conditions. In one embodiment the sufficient duration of H₂ production for an acute condition is approximately 30 minutes. For chronic, and in some embodiments, cosmetic condition conditions the sufficient duration is between 15-60 minutes. In other embodiments, however, the sufficient duration is between 6-8 hours. Those skilled in the art will understand that the duration of hydrogen delivery required for an ailment will be dependent upon that ailment. While some embodiments discuss duration for various accurate or chronic conditions, these time durations are for illustrative purposes only and should not be deemed limiting. The time duration to aid in healing of a specific ailment is dependent upon that ailment, the extensive nature of that ailment, the volume of hydrogen exposed, the location of the ailment, etc.

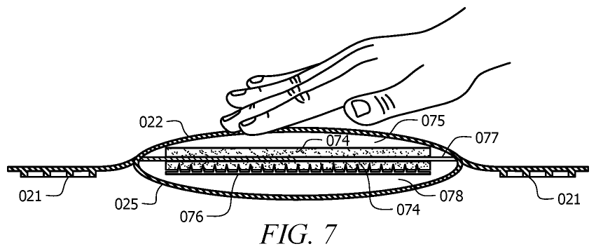

For safe and effective targeted delivery of H₂ to the body surfaces, the device is constructed of material impermeable for water and chemicals but permeable for hydrogen gas. The permeability and impermeability of the material, in some embodiments, is within the acceptable tolerance to those of ordinary skill. In one embodiment, the device is configured in such a way that the components capable of reacting with each other with the formation of H₂ are housed in separate compartments separated by a waterproof breakable partition and do not react with each other prior to the deliberate activation by the user. In order to activate the device for hydrogen production, the separating component, such as a baffle is destroyed by physical force, thereby releasing the contents of the inner compartments and initiating a hydrogen gas generating reaction. A separating component, as used herein, refers to any structure or arrangement which keeps and maintains components in a separated arrangement. The separating component can comprise a seal, a baffle, a wall, etc. In one embodiment the separating component comprises a destructible separating component. A destructible separating component is one which the separation ability of the separating component can be compromised or destroyed. As noted, in one embodiment the destructible separating component is destroyed by physical force which allows previously separated components to be in contact. In one embodiment, as an example, the destructible separating component comprises a capped capsule. The capsule can be broken or pierced to allow contact. In other embodiments, the capsule can comprise a cap/plug which is released when the capsule is pressed, thereby releasing the content into the outer compartment. In another design, the components of one partition are contained in an external vessel and are extruded into the other partition, thereby initiating hydrogen generating reaction. The layer between separate partitions is a separating component. In another design, depicted in FIGS. 7 and 8, the device comprises two compartments separated by a metal aluminum film laminated with a waterproof polymer film on the side facing the compartment containing liquid reactants. The aluminum side of the said partition is at the same time a reagent component that reacts with the liquid components in the other compartment when this partition is broken by indenting the grid with spikes. These variants of the design allows the user to store the device for an extended period of time before use. Following the reaction, H₂ generated in the device diffuses easily through the H₂-permeable film and penetrates the skin to promote healing at the desired location. In one embodiment, the chemicals inside the device remain in the package and are not in contact with the skin, thus minimizing hazardous and toxicity risks.

The activated device is applied to the target area of the body with a hydrogen-permeable side. The composition and amount of the chemicals is selected so as to maintain a supply of hydrogen to the target zone for certain periods of time, ranging from few minutes to several hours. During this time the hydrogen diffuses from within the sealed bag with an ongoing chemical reaction and is delivered to the tissue of the target body area. As noted above, the duration of the hydrogen supply depends on various factors, such as the volume of the bag and the amount of reagents, the kinetics of various chemical reactions, which depends on the reaction temperature, the nature and concentration of the chemicals used in the reactions, the presence of catalysts, and the permeability of the film in contact with the body surface. For example, the continuous supply of hydrogen formed by the reaction of borohydride with water can last for several minutes in some embodiments, while the production of hydrogen by the reaction of zinc metal powder with citric acid lasts 2-4 hours if the bag is applied to the surface of the body, thereby reaching body temperature. The kinetics of hydrogen production also depends on the addition of inert fillers such as cellulose, starch or agar, which will delay the diffusion of the reactive reactants and their mixing, thereby slowing down the reaction and thereby extending the duration of the hydrogen flow. Finally, the addition of cooling agents such as urea, which lowers the temperature due to its endothermic reaction when dissolved in water, slows down the hydrogen generation reaction. In some embodiments, the chemicals inside the device remain in the package and are not in contact with the skin, thus minimizing hazardous and toxicity risks.

In some embodiments, the device comprises adhesive tape on the top side extending over perimeter to tightly hermetically attach it to the body surface to prevent release of the hydrogen into atmosphere. In one embodiment, the hydrogen-permeable surface of the device is covered with a layer of oil (mineral oils, plant oils, such as linseed, olive, soybean oil, or their mixture) or other non-polar chemical and lipid-containing compounds, such as petro-gel, oil-containing ointments or creams to accumulate dissolved hydrogen gas for sustained delivery to the body tissue. In one embodiment, the concentration of hydrogen in the oil-based layer is greater than 4%.

The device allows continuing saturation of tissues with hydrogen and is applicable for cosmetic or medical purposes due to the known antioxidant, anti-inflammatory and anti-aging properties of hydrogen gas. In some embodiments, the device is disposable, does not require specific skills and can be easily self-administered.

The therapeutic hydrogen-generating devices, apparatuses, and methods for delivering molecular hydrogen to the desired body part are discussed herein. In the following descriptions, for purposes of explanation, numerous specific details are set forth in order to provide a thorough understanding of the present invention. It will be evident, however, to one skilled in the art that the present invention may be practiced without these specific details.

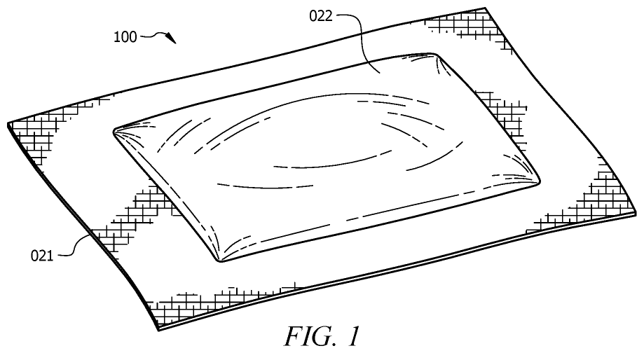

The present device will now be described by referencing the appended figures. In one embodiment, the device 100 as shown in FIG. 1 from a top view with the flexible outer layer film 022 which covers a portion of the top of the device 100. The flexible outer layer film 022 in one embodiment, comprises a material with significantly low permeability to the molecular hydrogen that the device will discharge. The film prevents the diffusion of hydrogen gas into adjacent space/air and at the same time ensures proper contact with the skin due to its adhesive properties. Examples of the outer film 022 include adhesive polymers used in medical device applications, which can include but are not limited to acrylic, epoxy resins, polyvinyl ester, polyvinyl chloride. The permeability of 1-2 mil thick outer films made of these materials is 10-100 times lower than a hydrogen-permeable skin-facing film of about the same thickness. For simplicity, the outer layer film with low permeability to hydrogen hereinafter referred to as the hydrogen-impermeable film.

The permeability of films depends on many factors, including the nature of the material used to prepare the film, temperature, pressure, time. Because the device is used at ambient temperature after activation, the parameters that determine permeability are time and internal pressure formed due to generation of hydrogen gas inside of the device. To consider a film impermeable, in some embodiments, the hydrogen diffusion coefficient of the film is approximately an order of magnitude lower than that of a permeable film (as mentioned in [0029]). Hydrogen gas impermeable film means that there is virtually no significant diffusion of hydrogen gas through the said film for the duration of the device's use after it has been activated (for several hours as indicated in [0023]). Examples include but not limited to polymeric materials commonly used for long term hydrogen storage such as high-density polyethylene, polyethylene terephthalate, polyamide, nylon, polyvinyl chloride and also adhesive polymers listed in [0029].

Hydrogen gas permeable film has properties characteristics that allow H₂ permeation through materials. “Permeable” film means that the transport of hydrogen gas through the specified film is efficient, which is determined by the internal pressure formed inside the device during the hydrogen formation reaction, and the chemical and structural characteristics of the films. In turn, the internal pressure depends on the rate of hydrogen diffusion, which is determined by the molecular structure of the polymer. The internal pressure depends on the rate of hydrogen formation during the chemical reaction after the activation of the device and its diffusion. In examples of the microporous material used to make the permeable layer 025, the internal pressure is close to atmospheric, since the generated hydrogen freely passes through the pores and is delivered to the tissues of the body. In the case of non-porous films, the process of hydrogen transport to the body is determined by the diffusion rate, which is determined by the properties of the material and internal pressure. Thus, the diffusion rate is determined by the kinetics of a chemical reaction and changes during the reaction, reaching its highest values when the gas production rate exceeds its diffusion rate. Examples of non-porous films that are considered permeable to H₂ include, but are not limited to, LDPE, PPE, silicone (as stated in [0040] as an example, polytetrafluoroethylene (PTFE), neoprene, natural rubber, whose permeability coefficients, expressed as volumes of hydrogen passing through a certain the area and thickness of the film vary in the range of 10 -300 compared to an impermeable film, which has permeability coefficients <1.

Thus, in one embodiment, an impermeable material is one where there is virtually no significant diffusion of hydrogen gas through the said film for the duration of the device's use after it has been activated.

As noted, in some embodiments, the device also has an adhesive, such as an adhesive tape on the top side extending over perimeter to tightly hermetically attach it to the body surface to prevent release of the hydrogen into atmosphere. As depicted adhesive 021 forms the perimeter of the device 100.

As noted, the outer side of the bag, which will be facing air, is laminated or sputtered with a hydrogen impermeable outer layer 022. In one embodiment the hydrogen impermeable outer layer 022 comprises aluminum. The hydrogen impermeable outer layer 022 prevents diffusion of hydrogen through this side and release into atmosphere. Thus, the side of the device which faces away from the body comprises a hydrogen impermeable layer. This ensures the produced hydrogen is directed to the desired location and not released into the air.

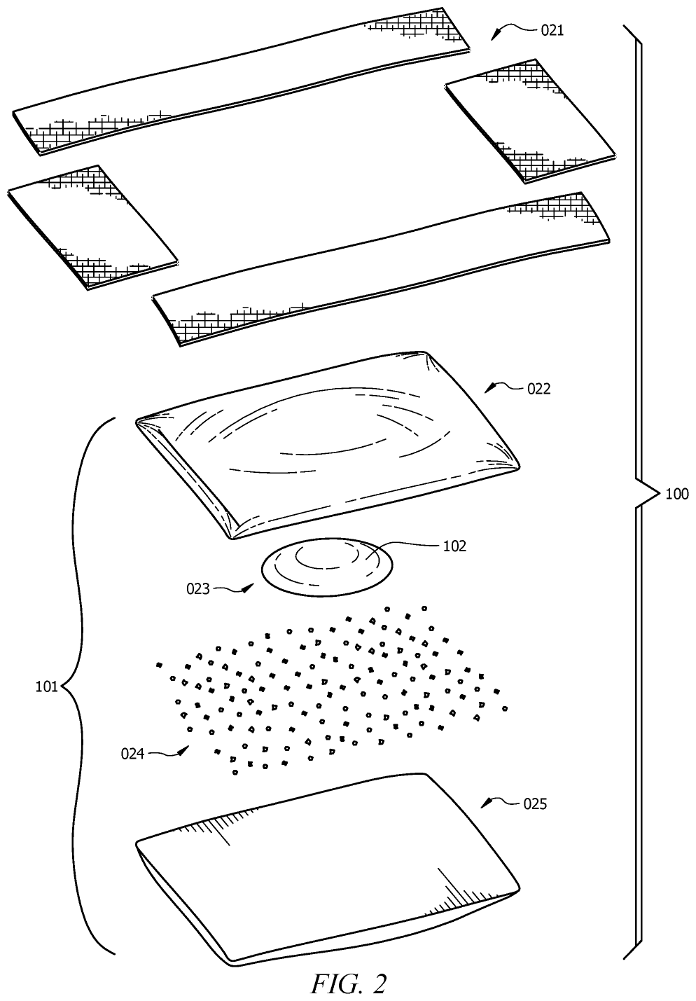

The exploded view of FIG. 2 further demonstrates the embodiment 1 of the device with the flexible container made of a hydrogen impermeable outer layer 022. As noted, the hydrogen impermeable outer layer 022 faces away from the body. The flexible device 100, as shown, comprises the hydrogen impermeable top outer layer 022, an adhesive 021 which forms the perimeter of the top outer layer. The device 100 further comprises a packet 023 which contains a solution. While the term “packet” is used, the packet can comprises a pouch, capsule, or virtually any other container for containing a solution. As noted above, in some embodiments, the packet 023 comprises a separating component. In some embodiments the packet 023 comprises a destructible separating component. The solution within the packet 023 is dept separate from chemicals 024. The chemicals 024 comprises a dry power in some embodiments. The packet 023 and the chemicals 024 are both stored beneath the top out layer 022.

While one embodiment shows the packet 023 housing solution and the chemicals 024 not housed, this is for illustrative purposes only. As an example, in some embodiments the chemicals 024 are also housed in a packet. In other embodiment only the chemicals 024 are housed in a packet. Thus, at least one of the chemicals 024 or the solution are housed in a packet. This is what allows the components to be separated.

The dry powder chemicals 024 are inert and non-reactive, until the solution containing packet is ruptured. In some embodiments, the dry chemical mixture 024 also contains an inert filler such as agarose powder or cellulose, which does not react after activation of the device. The filler compound maintains the reacting chemicals in a dispersed form and prevents aggregation. In addition, it helps to retain the added water or other solution inside the bag and maintain its shape. These specific fillers are provided for illustrative purposes only and should not be deemed limiting.

The solution and the chemicals 024 are sandwiched, in one embodiment, between the hydrogen impermeable top outer layer 022 and a hydrogen permeable bottom inner layer 025. The hydrogen permeable bottom inner layer 025, as described below, is adjacent to the body to provide delivery of hydrogen to the desired location.

The reaction between the dry powder chemicals 024 and the solution containing packet 023 only occurs after the separating component is ruptured. As noted, this can comprise splitting the packet 023, for example, such that the packet 023 can contact the chemicals 024, or vice versa. The subsequent reaction generates the molecular hydrogen. The molecular hydrogen then exits the container through the hydrogen permeable layer 025 that comes into contact with the body or skin.

The hydrogen permeable layer 025 can be made of various materials including, but not limited to, low density polyethylene (LDPE), polyphenylene ether (PPE), or silicone gas permeable films. These films can range in thickness. In one embodiment the hydrogen permeable layer 025 ranges from about 0.5 to 10 mil thick, depending on the desired H₂ permeability of the film. In addition, in some embodiments, the film thickness is selected based on breaking strength. Breaking strength is important when the components inside the bag can pose a moderate risk of skin irritation if they leak from the device. The permeable layer 025 can also comprise microporous polypropylene (PP) or polyethylene (PE). In some certain embodiments the permeable layer 025 comprises a through-pore size up to 0.45 μm, or perforated films, or a woven material such as KT tape. These types of skin-facing bottom layer allow reaction products to diffuse and be delivered to the body surface for cumulative with gaseous hydrogen beneficial effects on body tissues. As noted, in some embodiments, the hydrogen-permeable surface of the device 025 is covered with a layer of oil or other lipid containing compounds on side facing the body tissue to accumulate dissolved hydrogen gas for sustained delivery to the body tissue.

As shown in FIG. 2, the top outer layer 022 and the bottom inner layer 025 sandwiches the solution 102 and the dry chemicals 024. One or both of either the dry chemicals or the solution are maintained in a packet to keep the components separated until desired. The combination of the outer layer 022, solution 102, dry chemicals 024, and bottom inner layer 025 is referred to as a container 101. As shown, the adhesive 021 forms the perimeter around the container 101.

Thus, in one embodiment there is a device 100 for supplying hydrogen to a body surface. The device 100 has a dry chemical 024 separated from a solution 102. Both the dry chemical 024 and the solution 102 are housed in a container 101. The container 101 comprises a hydrogen impermeable top outer layer 022 and a hydrogen permeable bottom inner layer 025. The container 101 can further comprise an adhesive tape 021. As shown in FIG. 2, the adhesive tape 021 forms around the perimeter of the container 101.

In another embodiment, layer 046 is a pad soaked/impregnated with solutions or formulations that can accumulate/store hydrogen as well as provide cosmetic and therapeutic benefits. As an example, such a compound can be polysaccharide glycogen, which is able to accumulate hydrogen gas at higher concentrations than water. Glycogen has hydrating properties for the skin, so in addition to the benefits of hydrogen, the device has moisturizing properties and this variant of the device can be used for cosmetic purposes.

In another modification, layer 046 (FIG. 4) can contain compounds that not only facilitate the delivery of hydrogen to target tissues, but also allow the delivery of cosmetic or therapeutic agents for the treatment of skin lesions, as well as subcutaneous drug delivery. For example, such compounds are liposomes. Since hydrogen is highly soluble in the hydrophobic layers of liposomes, such particles will serve as a means of delivering hydrogen in combination with other cosmetic or therapeutic agents to tissues.

Various examples of hydrogen storage compounds or compositions producing hydrogen via reaction are provided below. These examples are not exhaustive and it would be readily apparent to those of ordinary skill in the art that other embodiments and examples are possible.

### EXAMPLE 1

In this version, the device 100 comprises a mixture of metallic aluminum powder and Ca(OH)₂ as the dry chemicals 024 inside a packet 023. Activation is achieved by releasing solution of NaCl from a second packet 023, which can comprise an insert or capsule. The solution is released and allowed to react with the powder chemicals 024 in the bag 022. Hydrogen is produced by a reaction of aluminum with water:

2Al+6H₂O→2Al(OH)₃+3H₂

Ca(OH)₂ and NaCl play a role of a reaction promoter by disrupting protective aluminum oxide layer. Where NaCl produces localized pitting and rupture of the alumina layer on aluminum surface, and Ca(OH)₂ dissolves existing alumina:

3Ca(OH)₂+Al₂O₃+3H₂O→Ca₃Al₂(OH)₁₂

One example will be provided with specific weights for the above reaction. This is for illustrative purposes only and should not be deemed limiting. The proportion of chemicals used in the reaction are as follows: aluminum powder—0.5 g; Ca (OH)₂—25 mg; the packet 023 contains 5% solution of NaCl with a volume of 16 mL. This proportion of the chemical components is suitable for a plastic bag 022 measuring 10 cm×10 cm.

In another variant, an aluminum foil sheet is used instead of powder. In one embodiment the sheet measures 9 cm×9 cm and has a weight of about 0.5 g. After the packet 023 is broken, the solution of NaCl is quickly spread on the surface of aluminum foil sheet, dissolves calcium hydroxide and initiates the reaction.

At these proportions of reagents, the reaction starts rapidly as evidenced by the appearance of small bubbles on the surface of aluminum foil 2-5 min after initiation of the reaction and is completed within 120 min. The generation of hydrogen rapidly increases within 5-10 min at ambient temperature reaching a plateau after ˜30 min. At body temperature (37° C.), the reaction develops faster, within 10-20 min. The composition and proportions of NaCl+Ca(OH)₂ in aluminum-based generation of hydrogen is safer than solely Ca(OH)₂, and runs under more controllable conditions. There is moderate pressure without developing a runaway or out-of-control condition if hydrogen permeable bottom inner layer is a perforated LDPE. In this specific embodiment, the maximum amount of hydrogen produced under these conditions is ˜0.6 L with an average flow in the range ˜2-5 mL/min.

CaO can be used instead of Ca(OH)₂. Upon activation, CaO will react with water producing Ca(OH)₂:

CaO+H₂O→Ca(OH)₂

This reaction is very exothermic (ΔH=−63.7 kJ/mol), thus adding a function of a heat pack to the hydrogen delivery. This brings an additional enhancement effect in treating injuries or damaged tissues, much the same way as heat packs are now used.

For a bag containing the inner packet 023 with 16 ml of 5% NaCl solution, the amount of CaO is 1.8 g to reach the temperature 50-55° C., which is reached in 1-2 min. Under these conditions, the subsequent reaction of aluminum with the production of hydrogen develops within 2-5 min reaching the plateau after 10 min.

### EXAMPLE 2

In this version, the device 100 contains a metallic powder or granules. Activation is achieved by releasing solution of acid from the packet 023. The metal in the chemical mixture 024 reacts with acid producing hydrogen. One example of this specific embodiment is shown below.

2Me+2HA→MeA+H₂

This specific example can be used to treat both chronic and cosmetic skin conditions. It can also be used for skin care.

In one of the variants of the device 100, metallic zinc powder is mixed with citric acid in the 1:5 molar ratio and placed in the device 100. When the inner packet 023 containing water is broken, reaction of zinc with citric acid is initiated:

3Zn+2C₃H₅O(COOH)₃=3Zn⁺²+2C₃H₅O(COOH)⁻₃+3H₂

For the device 100 measuring 10×10 cm, 20 mg of Zn and 600 mg of citric acid is used, which is dissolved in 6 ml H₂O when the inner packet 023 is broken thus bringing the contration of citric acid to ˜0.5 M. Under these conditions, the reaction is slow and continues for a few hours (6-8 h) under the body temperature until all metallic Zn is consumed with a maximum H₂ yield of 25 ml.

In another modification, the reaction proceeds for 1-2 h if sodium nitrate is added to the final concentration of 0.5 M when dissolved in water released from the packet 023. The dissolution of sodium nitrate in water in an endothermic reaction, which adds a cooling function to the device without negative effect on the release of hydrogen, thereby providing a synergistic effect of more efficient hydrogen production and cooling function in cases when the cooling pack is beneficial for the treatment of traumas and injuries.

In another variant of the device, metallic zinc powder is mixed with agar or another inert filler, such as cellulose in the proportion 1:1 by weight. The reaction of hydrogen production is initiated after release of HCl solution from the inner packet 023:

2Zn+2HCl=Zn⁺²+2Cl⁻+H₂

At the concentration of 160 mM HCl, added in excess of 5× by molarity reacts with Zn powder for 30-60 min at the body temperature with a rate of H₂ formation in the range 1-0.1 ml/min.

Other metals that can be used in the reaction with hydrochloric acid include reduced iron and magnesium.

In another embodiment, the skin facing inner layer film 025 can be permeable to the reaction products of zinc metal and organic or inorganic acids. Such a device 100 is useful for treating a variety of dermatological conditions, including skin infections, inflammatory lesions, neoplasms, and other disorders, as zinc salts are known to be therapeutic in such conditions. Thus, the combined effect of the topical administration of zinc salts and hydrogen will be achieved.

In another embodiment, the zinc metal reacts with the CuSO₄ solution released from the packet 023, resulting in two sequential reactions: a displacement reaction that produces ZnSO₄, and a reaction of an acidic copper sulfate solution with zinc to produce hydrogen gas. Zinc sulfate is widely used in dermatology for topical application in the form of 5-20% lotions in the treatment of a wide range of various skin lesions and diseases. Thus, the effects of ZnSO₄ and H₂ are additive or synergistic, as in the case of the treatment of skin inflammation.

Zn+CuSO₄→Cu+ZnSO₄ Reaction 1:

H₂SO₄+Zn+CuSO₄→H₂+Cu+ZnSO₄ Reaction 2:

The advantage of this embodiment is that the components and reaction products are non-toxic and safe for disposal if all the zinc metal powder is used up. This is achieved by using a solution of CuSO₄ in a molar excess.

### EXAMPLE 3

In this version, the device 100 contains metal borohydride (for example, sodium borohydride) as the dry powder chemicals 024. Activation is achieved by releasing water or other aqueous solutions from the packet 023 into container 101. The borohydride slowly reacts with water to generate molecular hydrogen:

NaBH₄+2H₂O→NaBO₂+4H₂

The reaction is exothermic and can also be used as a heat pack in combination with hydrogen therapy. In one embodiment, this combination is used to treat acute ailments.

### EXAMPLE 4

In another version the container 101 contains a metal silicide (Mg₂Si, Ca₂Si, FeSi) as the dry chemicals 024. Activation is achieved by releasing dilute solution of acid from the packet 023 into the container 101. The acid reacts with silicide and generates hydrogen:

Me₂Si+4HA(diluted)+2H₂O→2MeA₂+SiO₂+4H₂

In one embodiment, this combination of reactants is used to treat both acute and chronic ailments.

### EXAMPLE 5

In another version the container 101 contains a metal or alloy hydride (for example MgH₂, CaH₂, NaAlH₄, VHₓ, rare-earth metal alloys) as the dry chemicals 024 and water as the solution 102. Activation is achieved by releasing water from the packet 023 into the container 101. The hydride reacts with water and generates hydrogen:

MeH₂+2H₂O→Me(OH)₂+2H₂

In one embodiment, this combination of reactants is used to treat acute ailments.

### EXAMPLE 6

In another version the container 101 contains a metallic platinum catalyst as the dry chemicals 024 and formic acid and sodium formate as the solution 102. Activation is achieved by releasing the solution 102 from the packet 023 into the container 101. The platinum catalyzes decomposition of formic acid at ambient temperature and normal atmosphere pressure , which produces hydrogen:

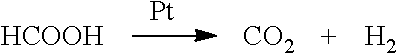

In one embodiment, this specific combination of reactants can be used to treat both acute and chronic ailments.

The list of the examples above is not exhaustive and is presented without loss of generality. The reactive components of the device may include any material used as a hydrogen storage, as long as they meet low toxicity requirements and are also cost effective. The main groups of such components are represented by hydrogen adsorbents, such as porous carbon-based materials or other hydrogen carrier molecules such as ammonia, formic acid, borohydrides, organic and metal hydrides in which hydrogen is released when such materials react with water or other aqueous solutions. Hydrogen-forming reactions can also occur during the decomposition of various organic and inorganic molecules (for example, formic acid), catalyzed using catalysts such as noble or rare metals (platinum, palladium, ruthenium or iridium) or hydrogenases in the case of enzymatic hydrogen formation reactions with the corresponding carbohydrate substrates. In all of the above cases, the choice of components and the design of the bag are determined by the degree of toxicity of the components and the properties and characteristics of the reactions, such as temperature and reaction rate, as well as the cost of the components. The choice is also based on the device's specific applications, safety and cost.

In one embodiment, a cooling function is added to versions described in above examples by adding ammonium nitrate, calcium ammonium nitrate, or urea to the mixture of dry chemicals 024. The cooling function is typically added for acute ailments, but this should not be deemed limiting. Upon activation these substances dissolve in water in an endothermic reaction, lowering the temperature of the bag. This reaction adds a function of a cool pack to the hydrogen delivery. This brings an additional enhancement effect in treating injuries or damaged tissues, much the same way as cool packs are now used. Since the hydrogen formation reaction will slow down as the temperature drops, the use of these additives will be particularly beneficial if the reaction proceeds too fast, as is the case with the reaction of bohrohydrides with water. In this case, the hydrogen supply will last longer time, which will provide more effective absorption by the skin, where the hydrogen concentration usually increases exponentially over 10-15 minutes and reaches a plateau after 40-60 minutes. It is necessary that the hydrogen-releasing bag fits snugly to the skin around the perimeter, otherwise saturation of the subcutaneous tissues with hydrogen is not achieved.

The top outer layer 022, shown in FIG. 1, can be labeled with thermochromic inks, allowing an individual to know that the device has been activated, as the temperature inside the device will change when the device is activated, depending on the composition of the reagents and the nature of the reaction (exothermic or endothermic).

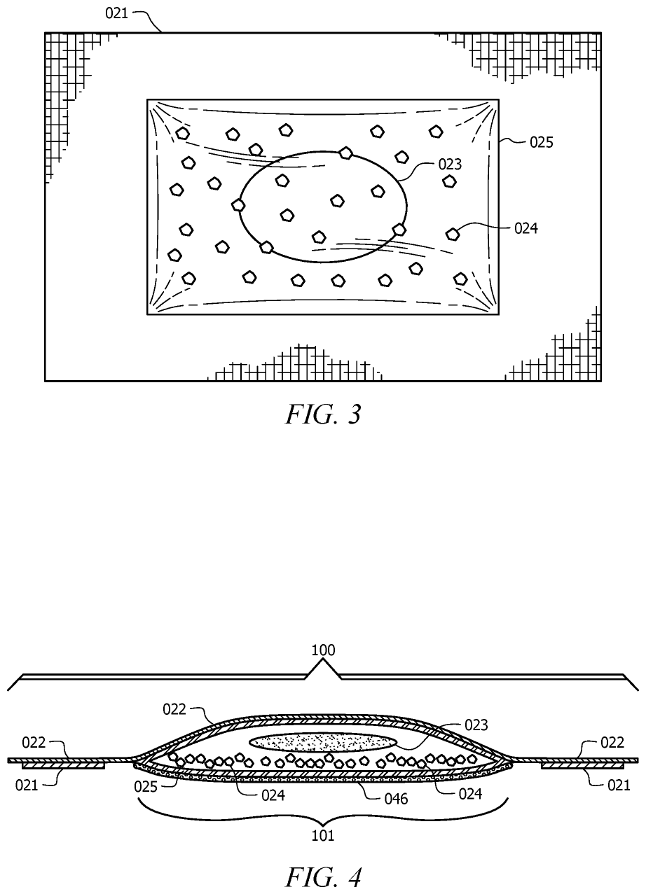

FIG. 3 illustrates a bottom view of an example of some of the components of the therapeutic hydrogen-generating device. In one embodiment, the hydrogen permeable bottom layer 025 is translucent to show the other components of the device. The dry chemical 024 and the solution containing packet 023 are visible in their inert stages, allowing an individual to know that they device has not been activated. This side of the device that will contact body surfaces can be lubricated with oils, petrogel, or other ointments and gels that will facilitate skin contact and provide additional beneficial effects, as previously discussed. Due to the high solubility of H₂ in oils, this type of lubricant will also store hydrogen gas at high concentrations and enhance the transdermal delivery of hydrogen gas across cellular phospholipid membranes.

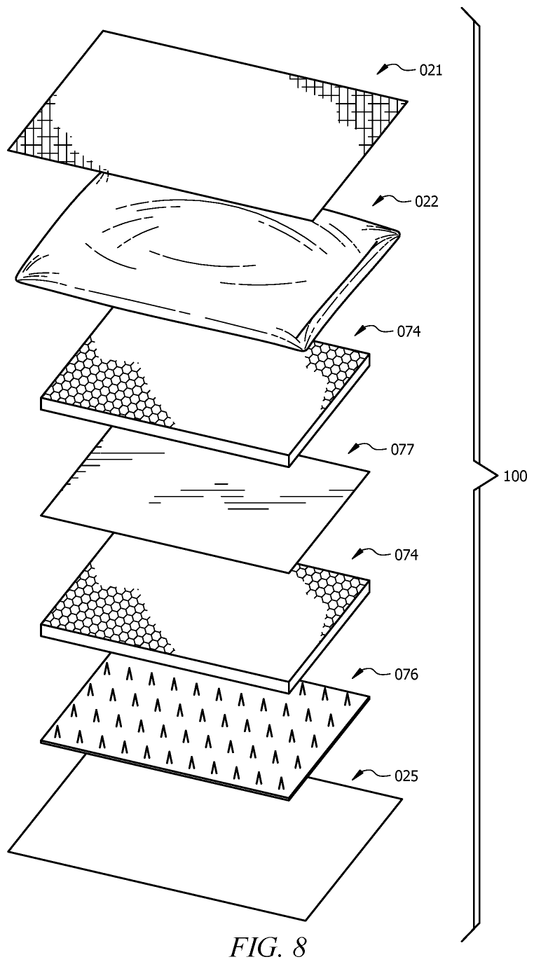

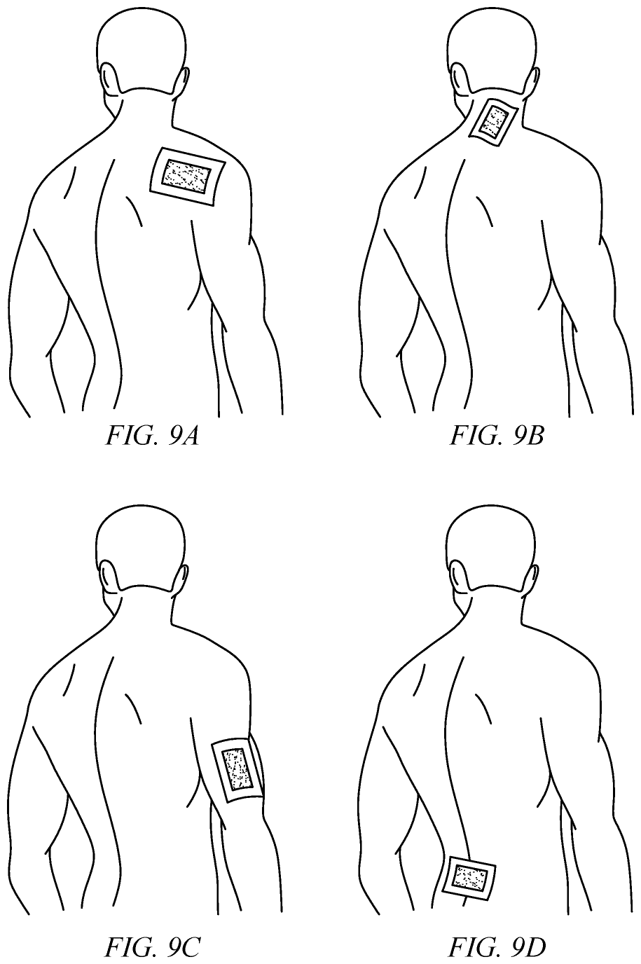

FIG. 4. depicts a perspective view of the elements that can comprise a therapeutic hydrogen-generating device 100 according to various embodiments of the present device. In one embodiment, each of the elements of the device are configured such that a flexible device 100 comprises an outer hydrogen impermeable top layer 022. The top layer 022 contains any produced molecular hydrogen and keeps it close to the desired body surface rather than leaking to the adjacent air where it would not be utilized as intended. The device 100 can further comprises a hydrogen-permeable material, e.g. a plastic covered on the top, atmosphere-facing side with hydrogen-impermeable material, such as aluminum foil layered over a thick plastic. The hydrogen permeable bottom inner layer 025 can also be made of a woven fabric, plastic (PVC, polyethylene or polyurethane), or latex strip. The flexible inner layer 025 can be lubricated with an oil-containing lotion 046, as shown, to facilitate saturation of the oil-containing lotion with hydrogen for improved diffusion into the target body tissue. One benefit of the device 100 is that placement on the body can be done in a variety of places as demonstrated by FIGS. 9A-D.

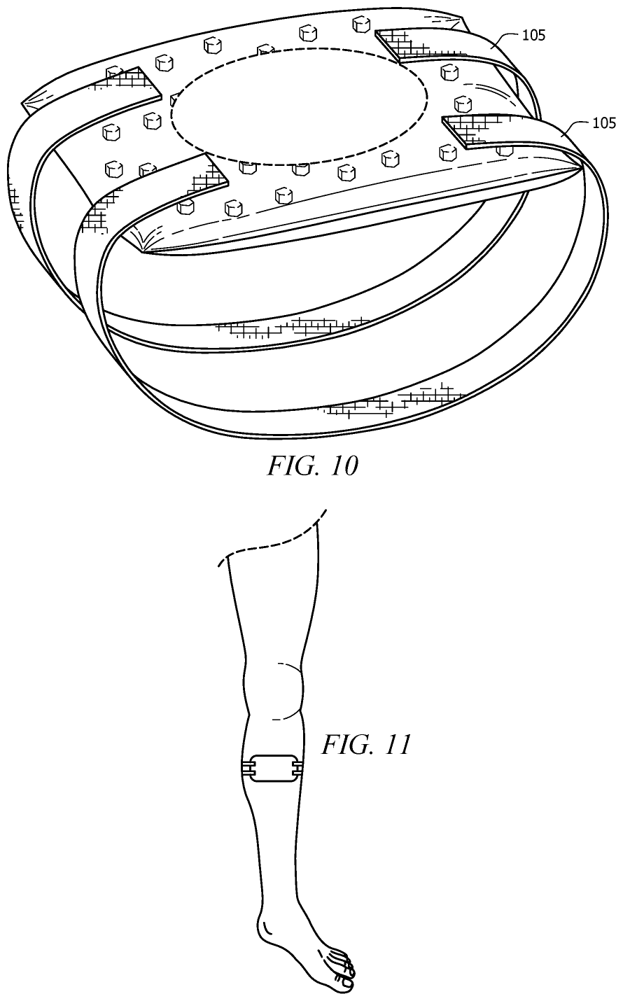

Another embodiment of the device is illustrated in FIG. 10 includes longer straps that can be used to wrap around an entire portion of the body (i.e. the leg or arm). This allows the device to stay on a specific location with a greater degree of success.

FIG. 11 shows the device wrapped around the calf of an individual with the longer straps keeping it in place.

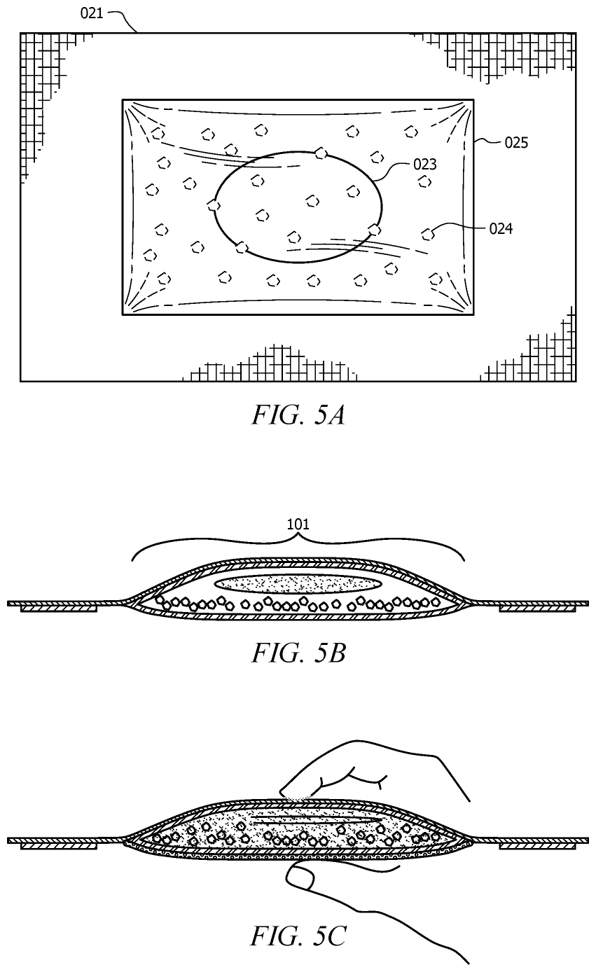

FIG. 5A illustrates a top view of a device in one embodiment. FIG. 5B shows a side view of an un-activated device in one embodiment. FIG. 5C shows a side view of an activated device in one embodiment. As noted, the top outer film 022 can be coated with a substance that is practically impervious to hydrogen, such as Mylar or sputtered aluminum. The permeability of such films to molecular hydrogen is substantially lower than that of skin-facing hydrogen-permeable films, allowing efficient delivery of hydrogen to the body surface/skin rather than venting it to the atmosphere. The outer film 022 also extends out till it joins the outer band, or adhesive 021 which can be a tape like substance to keep the device in place. The side of the adhesive 021, or outer band that faces towards a body part can have an adhesive that would help the device close to the skin. The container 101 holds the chemicals 024 and the packet 023 with the solution 102. The bottom layer of the container 101 comprises the hydrogen permeable surface 025 that allows for the molecular hydrogen to exit the container 101 to enter the body. Any of the permeable materials discussed herein can be utilized. The list of such materials is not limited to the above examples. For such purposes, any breathable and waterproof material can be used. In one embodiment the inert chemicals 024 and the solution 102 in the packet 023 can be activated by applying pressure as is shown in FIG. 5C to the entire container 101. The pressure breaks the capsule 023, releasing the solution into the chemicals 024. The resulting molecular hydrogen then flows out the container 101 through the hydrogen permeable membrane 025 to the desired body surface.

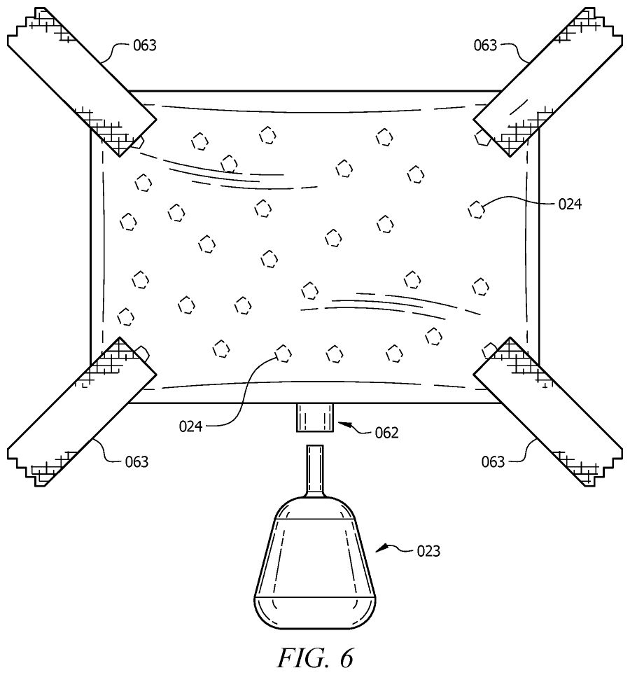

FIG. 6 is another example of an alternative embodiment of the device. As shown, the packet 023 is not contained in the container 101. The packet 023 introduces the solution into the container 101 through a port 062. The port 062 is on the side of the container 101 and the solution travels through the port into the container where the dry chemicals 024 are stored. The hydrogen is then produced and exits the device through the hydrogen permeable membrane 025, as previously described.

FIGS. 7-8 show another embodiment of the device. As shown, the device is constructed in the form of two compartments, a first compartment 075 and a second compartment 078. The compartments are separated by a septum 077. In one embodiment the septum 077 comprises an aluminum foil laminated with a waterproof polymer on the side facing the first compartment. The first compartment 075 houses liquid reactants. In one embodiment, the liquid reactants in the first compartment 075 is a mixture of NaCl solution and a suspension of Ca(OH)₂. The waterproof laminate film prevents the reagents in compartment 075 from coming into contact with metallic aluminum. Hydrogen is generated when the septum 077 is perforated by applying pressure to the device. The reaction is initiated quickly because perforating the aluminum film with the spikes 076 not only releases the reactants from compartment 075 into compartment 078, allowing the reaction described in [0046] to proceed, but also destroys the aluminum oxide layer, thus increasing pitting. The generated hydrogen then exits the device through the hydrogen permeable membrane 025, as previously described. The advantage of this variant of the device is that the laminated aluminum film simultaneously serves as a partition/septum and a reagent component, which makes its production less costly. Its use is also preferable in acute conditions due to the rapid generation of hydrogen.

The spikes 076 can comprise one or a plurality of point objects which can pierce the septum 077. In one embodiment the spikes 076 comprise a plastic grid which has a plurality of spikes. In one embodiment the spikes 076 are sufficient length to pierce the septum 077 but not the impermeable top layer 022.

The sponges 074 provide a means to house and hold the liquid. The sponges 074 can comprise virtually any non-reactive material. In one embodiment they are porous so as to hold liquid. The sponges also prevent the spikes 076 from breaking the outer films of the device. In some embodiments the thickness of the sponge is comparable to the length of the spikes 074. Thus, this allows for the safe breaking of the septum 077. As noted, in one embodiment the septum 077 comprises aluminum foil laminated on the side facing the liquid chemicals. In such embodiments, the aluminum foil is protected from the reactants due to the lamination. However, as noted, when the devices is pressed, the spikes break the laminated surface. This allows the reagents to enter the second compartment 078 where the reagents will contact the aluminum and react. A further advantage of this design is that the points of puncture of the aluminum film by the spikes 074 will be the nucleation sites. This allows the aluminum metal to react more efficiently with the reagents. Typically, metallic aluminum oxidizes and is covered with a thin film of aluminum oxide which prevents the reagents from easily reaching the aluminum. However, when the film breaks, the aluminum is exposed at the break points. This provides more surface area to enhance the reaction.

FIGS. 9A-9D illustrate the placement of the device 100 at various locations on the human body. This figure illustrates the versatility of the device 100.

FIG. 10 depicts an embodiment of system/device as a wrap that is placed around an entire portion of a body according to various embodiments described herein. The brace 105 can be wrapped around a limb, such as an arm, wrist, ankle, leg, etc. The brace 105 maintains contact of the device 100 with the desired limb.

FIG. 11 illustrates an embodiment wrapped around a leg of the system/device according to various embodiments described herein. FIG. 11 shows the device of FIG. 10 being deployed and utilized on a leg.

Now that a system for a device 100 has been described, a method of using the system will now be described in one embodiment. A method for producing molecular hydrogen which is transmitted transdermally to a desired body part begins with obtaining the device. In one embodiment the device has a container 101. The container houses a dry chemical and a solution which kept separately. They are kept separately so that the subsequent reaction can be controlled.

The container has a hydrogen impermeable top outer layer 022 and a hydrogen permeable bottom inner layer 025.

Next, the device is placed adjacent to the human tissue desired to be treated. The device is placed such that the permeable bottom inner layer 025 is adjacent the human issue.

Thereafter, the reaction is initiated. In one embodiment initiating the reaction comprises at least partially destructing the container. This causes the dry chemical and solution to be mixed together. The destruction can take many forms including applying pressure to burst a packet 023 which can house the solution 102 or the dry chemical 024. Further, the destruction can take the form of breaking a cap, removing a cap, etc. Any force which causes the dry chemical 024, or dry chemicals, to mix with the solution 102 can be utilized.

Once the reaction is initiated, hydrogen gas expands and exits the container through the hydrogen permeable bottom inner layer. Because the top layer is impermeable, the only way for the gas to escape is through the permeable bottom inner layer. As the reaction continues, the pressure within the container 101 builds. To relieve the pressure, the produced hydrogen gas escapes through the permeable bottom inner layer 025 and into the adjacent human tissue.

The developed methods and device variants presented herein allows to overcome many of the shortcomings that suffer from the existing devices and methods for topical application and treatment of the body surface, as well as transdermal delivery of hydrogen to subcutaneous tissues. These include efficiency of the H2 delivery, sufficient duration, cost, availability for home use without the purchase of expensive hydrogen production equipment, and low health and toxicity risks.

The least toxic methods of transdermal delivery of hydrogen include the soaking hydrogen-rich water bath methods. However, this approach requires the preparation of hydrogen water in large volumes, which is very expensive and requires industrial scale electrolyzers and also high-concentrated hydrogen water generators. Therefore, hydrogen-water bathing therapy is possible only in medical settings. In addition, the solubility of hydrogen in water is low, about 1.6 mg/l at ambient temperature, so the soaking method does not achieve adequate therapeutic concentrations of hydrogen in body tissues. Even if high content of dissolved hydrogen is achieved by using specific equipment, it requires freshly prepared hydrogen water before immersing whole body in the hydrogen-water. Furthermore, such treatment can be conducted within relatively short period of time, as hydrogen quickly evaporates from the hydrogen-rich water, typically within 15-30 min.

Similarly, applying or spraying hydrogen directly to the skin is insufficient. Such an approach does not achieve long-term exposure to hydrogen, as hydrogen-enriched water sprayed onto the surface of the body will be rapidly depleted of hydrogen gas, and the hydrogen will mostly be released into the atmosphere.

The system and method discussed herein offers a simple solution which is contrasted to highly technical solutions requiring hydrogen tanks, etc. The device 100 is portable and safe.

The device and method proposed here has a number of advantages: 1) allows topical application to target areas of the body; 2) allows to maintain hydrogen concentration in target tissues at the saturation level for significant periods of time, from 30 minutes to several hours; 3) allows you to supply hydrogen dissolved in the oil/lipid-based coating with a concentration exceeding the maximum solubility in water up to 100 times (as an example the embodiment in paragraph 0026); 4) allows hydrogen to be delivered through a pad impregnated/soaked with substances concentrating hydrogen and facilitating the absorption of hydrogen by the skin and subcutaneous tissues, as well as providing beneficial cosmetic and therapeutic effects; 5) the device can be activated at any time and applicable by any person at home and does not require special equipment for and means for preparation of compositions with high concentrations of hydrogen gas; 6) the device, and variants allows the delivery of hydrogen in combination with other therapeutic agents, such as the reaction product ZnSO₄; 7) The use of the hydrogen-impermeable adhesive around the perimeter of the device prevents the release/seepage of hydrogen into the atmosphere and thus maintain a constant level of hydrogen saturation in tissues for prolonged periods of time; 8) The device allows cooling and heating effects; 9) The device is cost-efficient, the estimated price for a medium-sized bag (FIGS. 9-11) is from 2 to 10 dollars per device depending on the internal content, reagents/components and material/plastic/fabric.

Molecular hydrogen possesses strong antioxidant and anti-inflammatory properties and thus can be administered as a cosmetic or therapeutic mean for the treatment of many pathological conditions, especially those associated with oxidative stress and inflammation. Which method and device configuration options to use depends on the lesions and conditions to be treated. For example, for the treatment of acute conditions such as burns and sunburns, skin irritation, allergic reactions and other hypersensitivity reactions, bruising, swelling due to trauma, and other conditions that lead to an inflammatory response and increased oxidative stress, the best option would be to use variants of the device with faster H₂ formation reactions, as described examples above, as well as devices with facilitated diffusion of H₂, which depends on the characteristics of the H₂ permeable film adjacent to the body. For the treatment of back pain due to nerve inflammation, as well as hematomas and edema caused by trauma, the best option in the first two days post-injury would be the device versions directed to acute treatment with a cooling function. A variant of the device is also suitable for such purposes, in which the inner skin-facing film is made of woven fabric or microporous/perforated polymers that allow chemicals useful for the treatment to leak out. After 2-3 days post-injury, as well as for the treatment of pain in the joints, muscles and back pain, the best option would be the versions of the device described in paragraphs 0048 and 0062 with a heating function, in some embodiments.

In the case of chronic conditions such as atopic dermatitis, eczema, arthritis, muscle inflammation, which are associated with chronic inflammation, options discussed above with chronic ailments with a heating function, as well as options with a slower release of hydrogen, such as paragraphs 0053, 0054, 0056, 0064, 0068 will be the best as they allow longer exposure to hydrogen, for hours or even overnight, in some embodiments.

For cosmetic purposes, the best options, in some embodiments, would be device variants that use hydrogen in combination with factors beneficial to skin health, such as zinc sulfate, described in paragraph 0060, oil-based compositions or glycogen, such as described in paragraphs 0026 and 0043.

While the invention has been particularly shown and described with reference to a preferred embodiment, it will be understood by those skilled in the art that various changes in form and detail may be made therein without departing from the spirit and scope of the invention.

What is claimed is:

1. A device for supplying hydrogen to a body surface, said device comprising:
a dry chemical separated from a solution;
said dry chemical and said solution housed in a container;
wherein said container comprises a hydrogen impermeable top outer layer;
wherein said container comprises a hydrogen permeable bottom inner layer.

2. The device of claim 1 wherein said container comprises a destructible separating component.

3. The device of claim 1 wherein said solution is stored in a destructible packet.

4. The device of claim 1 wherein a hydrogen-producing reaction is initiated by destructing at least a portion of the container such that the dry chemical and solution can be mixed.

5. The device of claim 4 wherein when said dry chemical and solution are mixed, the mixing initiates a chemical reaction to produce hydrogen gas, and wherein said impermeable top outer layer results in virtually no significant diffusion of hydrogen gas through the said impermeable top outer layer during the duration of said chemical reaction.

6. The device of claim 5 wherein the hydrogen permeable bottom inner layer is in contact with body tissue, and wherein hydrogen gas diffuses through the hydrogen permeable bottom inner layer into the body tissue.

7. The device of claim 1 wherein said hydrogen impermeable top outer layer is laminated or sputtered with aluminum to prevent release of hydrogen into atmosphere.

8. The device of claim 1 further comprising an adhesive.

9. The device of claim 1 wherein said hydrogen permeable bottom inner layer is lubricated with an oil-containing lotion to facilitate saturation of the oil-containing lotion with hydrogen for improved diffusion into a target body tissue.

10. A method for producing molecular hydrogen that is transmitted transdermally to a desired body part, comprising the steps of:
a) obtaining a container housing a dry chemical and a solution which are housed separately, wherein said container comprises a hydrogen impermeable top outer layer, and wherein said container comprises a hydrogen permeable bottom inner layer;
b) placing said hydrogen permeable bottom inner layer against human tissue;
c) at least partially destructing said container to mix said dry chemical with said solution;
d) producing hydrogen via a reaction between said dry chemical and said solution;
e) directing said hydrogen through said hydrogen permeable bottom inner layer to said human tissue.

11. The method of claim 10 wherein said container is coupled to an adhesive.

12. The method of claim 10 wherein said container is coupled to a brace.

13. The method of claim 10 wherein said solution is housed in a destructible packet.

14. A device for supplying hydrogen to a body surface, said device comprising:
a solution housed in a first compartment;
a second compartment below said first compartment, wherein said first and second compartment are separated by a septum, wherein said septum comprises a non-reactive surface facing said first compartment which keeps said solution from reacting with said septum;
wherein said second compartment comprises a plurality of spikes for piercing said septum;
wherein said first compartment comprises a hydrogen impermeable top outer layer, and wherein said second compartment comprises a hydrogen permeable bottom layer.

15. The device of claim 14 wherein said first compartment comprises a sponge, and wherein said first compartment comprises a NaCl solution and a suspension of Ca(OH)₂.

16. The device of claim 14 wherein said septum comprises aluminum foil which is laminated on a side facing the first compartment.

17. The device of claim 14 wherein said second compartment comprises a sponge.

18. The device of claim 14 wherein when said spikes pierce said septum, said spikes form nucleation sites on said septum which allow said septum to react more efficiently with the reagents.
